(19) United States
(12) Patent Application Publication
(10) Pub. No.: US 2026/0108712 A1
(43) Pub. Date: Apr. 23, 2026

(54) MULTI-PART MEDICAL DEVICES WITH UNIDIRECTIONAL ROTATIONAL LOCKING MECHANISM

(71) Applicant: BOSTON SCIENTIFIC SCIMED, INC., Maple Grove, MN (US)

(72) Inventors: Sarah Tian-Hong Hua, Plymouth, MN (US); Ryan Robert Davis, Plymouth, MN (US)

(73) Assignee: BOSTON SCIENTIFIC SCIMED, INC., Maple Grove, MN (US)

(21) Appl. No.: 19/365,875

(22) Filed: Oct. 22, 2025

Related U.S. Application Data
(60) Provisional application No. 63/710,711, filed on Oct. 23, 2024.

(51) Int. Cl. A61M 29/02; A61M 25/00

(52) U.S. Cl. CPC A61M 29/02; A61M 25/0097; A61M 2210/125

(57) ABSTRACT

A variety of medical procedures utilize two or more medical device components, including medical device components where a first medical device component is advanced within a second medical component. An example would be an elongate dilator used in combination with a guide catheter. The guide catheter and the elongate dilator may be adapted to provide a unidirectional rotational lock mechanism that allows the guide catheter and the elongate dilator to lock together in only one relative rotational orientation such that rotating one of the components causes rotation of both components.

### CROSS REFERENCE TO RELATED APPLICATIONS

This application claims the benefit of priority of U.S. Provisional Application No. 63/710,711 filed Oct. 23, 2024, the entire disclosure of which is hereby incorporated by reference.

### TECHNICAL FIELD

The disclosure relates generally to medical devices and more particularly to medical devices having a locking mechanism that releasably provides a unidirectional rotational lock between components.

### BACKGROUND

A variety of medical procedures utilize two or more medical device components, including medical device components where a first medical device component is advanced within a second medical component. An example would be an elongate dilator used in combination with a guide catheter. There may be a desire to provide and maintain a particular rotational orientation between a curved region of the elongate dilator and a curved region of the guide catheter. In some instances, there may be a desire to be able to control the movement of both the first medical device component and the second medical device component without having to physically manipulate both components simultaneously. In some instances, there may be a desire to maintain a unidirectional rotational arrangement between the first medical device component and the second medical device component, meaning that only one rotational arrangement between the first medical device component and the second medical device component is permitted, and that when a physician or other professional rotates one component, the other component rotates as well. There is an ongoing need for improved medical devices and medical device systems.

### SUMMARY

This disclosure provides design, material, manufacturing method, and use alternatives for medical devices. An example may be found in a medical device assembly for accessing a left atrial appendage. The medical device assembly includes a first elongate medical device and a second elongate medical device. The first elongate medical device includes a first elongate shaft having a first proximal region and a first distal region and a first proximal hub that is secured to the first proximal region. The second elongate medical device includes a second elongate shaft having a second proximal region and a second distal region and a second proximal hub that is secured to the second proximal region. The first elongate medical device is adapted to form a releasable unidirectional rotational lock with the second elongate medical device.

Alternatively or additionally, the second elongate shaft may be adapted to fit within a lumen extending through the first elongate shaft.

Alternatively or additionally, the second proximal hub may be adapted to releasably couple to the first proximal hub when the second elongate shaft is disposed within the lumen extending through the first elongate shaft.

Alternatively or additionally, the second proximal hub may be adapted to form a releasable unidirectional rotational lock with the first proximal hub.

Alternatively or additionally, the first elongate medical device may be adapted to be advanced over a guidewire in order to reach an atrial septum.

Alternatively or additionally, the second elongate medical device may be adapted to be advanced over the guidewire in combination with the first elongate medical device in order to create or enlarge an aperture formed in the atrial septum.

Alternatively or additionally, the first proximal hub may include an aperture forming a first pattern and the second proximal hub may include an extension member that is adapted to extend into the aperture and that includes a second pattern complementary to the first pattern.

Alternatively or additionally, the first pattern and the second pattern may be adapted to only allow the extension member to fit into the aperture in a single orientation relative to the first proximal hub.

Alternatively or additionally, the first elongate medical device may include a guide catheter.

Alternatively or additionally, the second elongate medical device may include an elongate dilator.

Alternatively or additionally, the second proximal hub may include a graspable profile to facilitate advancement of the second elongate medical device into the first elongate medical device.

Another example may be found in a medical device assembly for accessing a left atrial appendage. The medical device assembly includes a guide catheter and a dilator. The guide catheter includes a guide catheter shaft and a guide catheter hub that is secured to a proximal region of the guide catheter shaft. The dilator includes a dilator shaft and a dilator hub that is secured to a proximal region of the dilator shaft. The dilator can only be inserted into the guide catheter with the dilator hub in a specific rotational orientation relative to the guide catheter hub.

Alternatively or additionally, the guide catheter hub may further include a threaded knob that when rotated is adapted to urge the dilator further distally after the dilator has been inserted into the guide catheter with the dilator hub in the specific rotational orientation relative to the guide catheter hub.

Alternatively or additionally, the guide catheter hub may further include an aperture adapted to accommodate the dilator hub, the aperture including one or more wings extending radially outwardly from the aperture.

Alternatively or additionally, the dilator hub may further include an extension member having one or more tabs that extend radially outwardly from the extension member, the one or more tabs complementary to the one or more wings.

Alternatively or additionally, the guide catheter may be adapted to be advanced over a guidewire in order to reach an atrial septum.

Alternatively or additionally, the elongate dilator may be adapted to be advanced over the guidewire in combination with the guide catheter in order to create or enlarge an aperture formed in the atrial septum.

Alternatively or additionally, the dilator hub may include a graspable profile to facilitate advancement of the dilator into the guide catheter.

Another example may be found in a medical device assembly for accessing a left atrial appendage. The medical device assembly includes a guide catheter and a dilator. The guide catheter includes a guide catheter shaft and a guide catheter hub that is secured to a proximal region of the guide catheter shaft, the guide catheter hub including an aperture having one or more wings extending radially outwardly from the aperture. The dilator includes a dilator shaft, a dilator hub that is secured to a proximal region of the dilator shaft, and an extension member that extends distally from the dilator hub. The extension member includes one or more tabs extending radially outwardly from the extension member that are adapted to fit into the one or more wings.

Alternatively or additionally, the one or more wings and the one or more tabs may be adapted to permit the extension member to fit into the aperture in only one relative rotational orientation between the guide catheter hub and the dilator hub.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 provides an illustrative example of part of a procedure for implanting an LAAC (left atrial appendage closure) device, which includes a guide catheter used in combination with an elongate dilator;

FIG. 2 is a schematic view of a medical device assembly including a first elongate medical device such as a guide catheter and a second elongate medical device such as a dilator;

FIG. 2A is a cross-sectional view taken along the line 2A-2A of FIG. 2;

FIG. 3 is a schematic view of the medical device assembly of FIG. 2, with the second elongate medical device shown engaged with the first elongate medical device;

FIG. 4 is a cross-sectional view taken along the line 4-4 of FIG. 3;

FIG. 5 is a partially exploded view of a first proximal hub forming part of the medical device assembly of FIG. 2;

FIG. 6 is an exploded view of the first proximal hub of FIG. 5; and

FIG. 7 is an exploded view showing a keyed base that forms part of the first proximal hub and an extension member that forms part of the second proximal hub, showing how the keyed base and the extension member combine to provide a unidirectional rotational lock between the first elongate medical device and the second elongate medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A variety of medical procedures may include the use of two different medical devices that are used in combination. As an example, a first medical device may be used in combination with a second medical device in order to gain access to a particular treatment site and then to perform an appropriate treatment at the particular treatment site. In some cases, a guidewire may be advanced through the vasculature of a patient to reach a particular treatment site. A first medical device may be advanced over the guidewire, and then a second medical device may be advanced through the first medical device. In some instances, there may be a desire to be able to advance and control the first medical device and the second medical device as an assembly, meaning that both the first medical device and the second medical device may be advanced or withdrawn axially without the user being required to separately manipulate both the first medical device and the second medical device at the same time. In some instances, the first medical device and the second medical device may be rotated as an assembly, meaning that the first medical device and the second medical device may be rotated together without the user being required to separately manipulate both the first medical device and the second medical device at the same time. In some instances, there can be advantages to being able to advance or rotate the first medical device and the second medical device together with the second medical device in a certain rotational orientation relative to the first medical device, particularly when the first medical device and the second medical device have curved distal regions and/or are steerable.

An illustrative but non-limiting example of a medical procedure that may utilize a first medical device and a second medical device in combination may include obtaining access to the left atrium of a patient to implant a left atrial appendage closure (LAAC) device. In particular, a first medical device such as a guide catheter may be used in combination with a second medical device such as an elongate dilator. It will be appreciated that two such medical devices may be used in combination in performing any of a variety of different medical procedures. Gaining access to the left atrium in order to deliver and implant an LAAC is merely an example of using a first medical device in combination with a second medical device.

In some instances, a medical device assembly is adapted for accessing a left atrial appendage. The medical device assembly includes a first elongate medical device and a second elongate medical device. The first elongate medical device includes a first elongate shaft having a first proximal region and a first distal region and a first proximal hub that is secured to the first proximal region. The second elongate medical device includes a second elongate shaft having a second proximal region and a second distal region and a second proximal hub that is secured to the second proximal region. The first elongate medical device is adapted to form a releasable unidirectional rotational lock with the second elongate medical device.

In some cases, the second elongate shaft may be adapted to fit within a lumen extending through the first elongate shaft. In some cases, the second proximal hub may be adapted to releasably couple to the first proximal hub when the second elongate shaft is disposed within the lumen extending through the first elongate shaft. The second proximal hub may be adapted to form a releasable unidirectional rotational lock with the first proximal hub. In some cases, the first elongate medical device may be adapted to be advanced over a guidewire in order to reach an atrial septum. In some cases, the second elongate medical device may be adapted to be advanced over the guidewire in combination with the first elongate medical device in order to create or enlarge an aperture formed in the atrial septum. In some cases, the first proximal hub may include an aperture forming a first pattern and the second proximal hub may include an extension member that is adapted to extend into the aperture and that includes a second pattern complementary to the first pattern. As an example, the first pattern and the second pattern may be adapted to only allow the extension member to fit into the aperture in a single orientation relative to the first proximal hub. In some cases, the first elongate medical device may include a guide catheter, and the second elongate medical device may include an elongate dilator. In some cases, the second proximal hub may include a graspable profile.

In some instances, a medical device assembly is adapted for accessing a left atrial appendage. The medical device assembly includes a guide catheter and a dilator. The guide catheter includes a guide catheter shaft and a guide catheter hub that is secured to a proximal region of the guide catheter shaft. The dilator includes a dilator shaft and a dilator hub that is secured to a proximal region of the dilator shaft. The dilator can only be inserted into the guide catheter with the dilator hub in a specific rotational orientation relative to the guide catheter hub.

In some cases, the guide catheter hub may further include a threaded knob that when rotated is adapted to urge the dilator further distally after the dilator has been inserted into the guide catheter with the dilator hub in the specific rotational orientation relative to the guide catheter hub. In some cases, the guide catheter hub may further include an aperture adapted to accommodate the dilator hub, the aperture including one or more wings extending radially outwardly from the aperture. The dilator hub may further include an extension member having one or more tabs that extend radially outwardly from the extension member, the one or more tabs complementary to the one or more wings. In some cases, the guide catheter may be adapted to be advanced over a guidewire in order to reach an atrial septum. In some cases, the elongate dilator may be adapted to be advanced over the guidewire in combination with the guide catheter in order to create or enlarge an aperture formed in the atrial septum. In some cases, the dilator hub may include a graspable profile.

In some instances, a medical device assembly is adapted for accessing a left atrial appendage. The medical device assembly includes a guide catheter and a dilator. The guide catheter includes a guide catheter shaft and a guide catheter hub that is secured to a proximal region of the guide catheter shaft, the guide catheter hub including an aperture having one or more wings extending radially outwardly from the aperture. The dilator includes a dilator shaft, a dilator hub that is secured to a proximal region of the dilator shaft, and an extension member that extends distally from the dilator hub. The extension member includes one or more tabs extending radially outwardly from the extension member that are adapted to fit into the one or more wings. In some cases, the one or more wings and the one or more tabs may be adapted to permit the extension member to fit into the aperture in only one relative rotational orientation between the guide catheter hub and the dilator hub.

The above summary of some embodiments is not intended to describe each disclosed embodiment or every implementation of the present disclosure. The Figures, and Detailed Description, which follow, more particularly exemplify these embodiments.

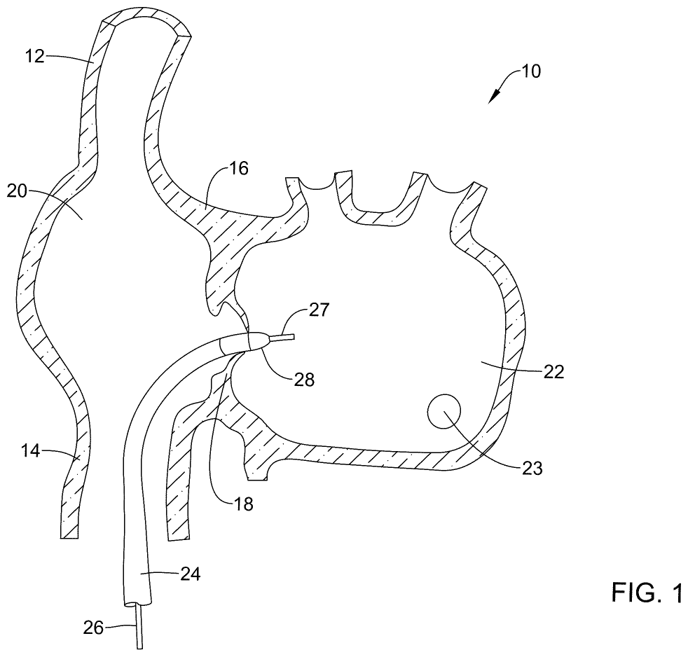

FIG. 1 provides a schematic view of a portion of a mammalian heart 10, including a superior vena cava 12, an inferior vena cava 14, a septum 16, an atrial septum 18, a right atrium 20 and a left atrium 22. In some cases, the left atrium 22 may include a left atrial appendage (LAA) 23. A composite medical device including a guide catheter 24 may be advanced over a guidewire 26. In some cases, the guidewire 26 may be an RF guidewire that is adapted to utilize RF (radio frequency) energy to cauterize, using a cautery tip 27. In some cases, for example, an RF guidewire may be used to form a small aperture in or near the atrial septum 18. In some cases, a guidewire may have a sharp distal end that may be used to form a puncture.

The puncture through the atrial septum 18 can be done from a position within the right atrium 20. By forming an aperture through the atrial septum 18, it is possible to reach the left atrium 22 from the relative safety of the right side of the heart. In some cases, an elongate medical device including an elongate dilator 28 may be advanced over the guidewire 26, and within the guide catheter 24. Once an aperture has been formed within the atrial septum, the elongate dilator 28 may be advanced over the guidewire and through the aperture in order to widen the aperture. From there, the elongate dilator 28 and guidewire 26 may be removed in order to allow a delivery device carrying an LAAC (left atrial appendage closure) device to be advanced through the guide catheter 24. A variety of devices may be advanced through the guide catheter 24 in order to reach the LAA 23. An illustrative example of a suitable LAAC device includes the Watchman™ FLX and Watchman™ FLX PRO devices commercially available from Boston Scientific Corporation of Marlborough, MA, United States.

In some cases, an assembly including the guide catheter 24 and the elongate dilator 28 may be advanced through the inferior vena cava 14 in order to reach the right atrium 20. It will be appreciated that this can represent a tortuous path through the vasculature. In some cases, the guide catheter 24 and/or the elongate dilator 28 may be adapted to have a curved distal end in order to facilitate steering.

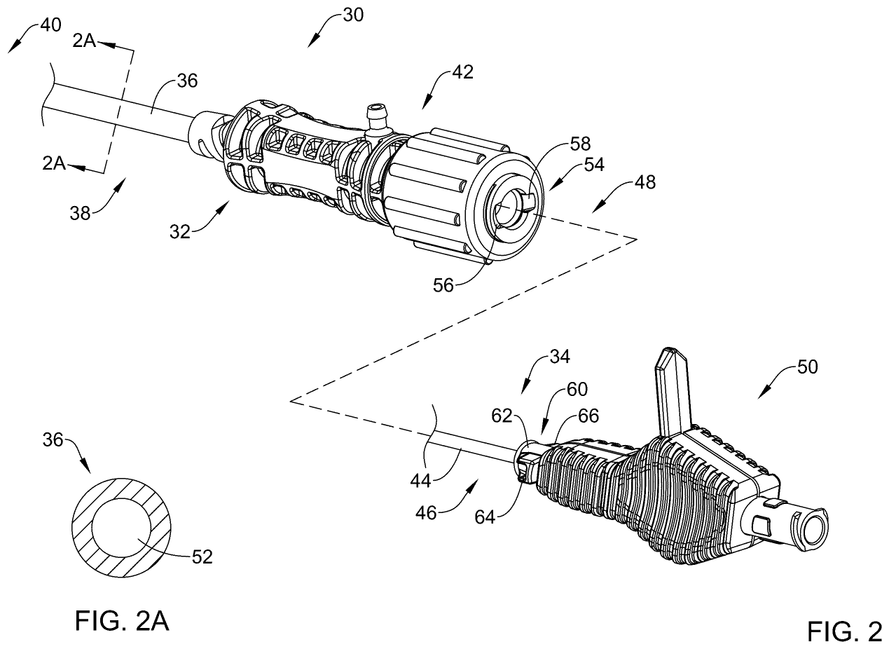

FIG. 2 is an exploded view of an illustrative medical device assembly 30 that includes a first elongate medical device 32 and a second elongate medical device 34. In some cases, the first elongate medical device 32 may be an example of the guide catheter 24 shown in FIG. 1. In some cases, the second elongate medical device 34 may be an example of the elongate dilator 28 shown in FIG. 1. The first elongate medical device 32 includes a first elongate shaft 36 that extends from a first proximal region 38 towards a first distal region 40. A first proximal hub 42 is secured to the first proximal region 38 of the first elongate shaft 36. The second elongate medical device 34 includes a second elongate shaft 44 that extends from a second proximal region 46 to a second distal region 48. A second proximal hub 50 is secured to the second proximal region 46 of the second elongate shaft 44.

The first elongate medical device 32 is adapted to form a releasable unidirectional rotational lock with the second elongate medical device 34. A unidirectional rotational lock means that the first elongate medical device 32 and the second elongate medical device 34 are, in combination, adapted to allow the second elongate medical device 34 to engage with the first elongate medical device 32 in only one relative orientation between the first elongate medical device 32 and the second elongate medical device 34. This means that any curvature present within the first distal region 40 of the first elongate shaft 36 and any curvature present within the second distal region 48 of the second elongate shaft 44 are able to be repeatedly arranged in a desired orientation with respect to each other. In some cases, having a unidirectional rotational lock between the first elongate medical device 32 and the second elongate medical device 34 means that a physician or other professional is able to manipulate the medical device assembly 30 as a unitary member, rather than as separate first and second elongate medical devices 32 and 34.

FIG. 2A is a cross-sectional view taken along the line 2A-2A of FIG. 2, and shows a cross-section taken through the first elongate shaft 36. As can be seen, the first elongate shaft 36 defines a lumen 52 extending through the first elongate shaft 36. In some cases, the second elongate shaft 44 may be adapted to fit within the lumen 52 that extends through the first elongate shaft 36. Returning to FIG. 2, in some cases, the second proximal hub 50 may be adapted to releasably couple to the first proximal hub 42 when the second elongate shaft 44 is disposed within the lumen 52 extending through the first elongate shaft 36. In some cases, the first elongate medical device 32 may be adapted to be advanced over a guidewire such as the guidewire 26 (FIG. 1) in order to reach the atrial septum 18 (FIG. 1). In some cases, the second proximal hub 50 may be adapted to be form a releasable unidirectional rotational lock with the first proximal hub 42. As an example, the second elongate medical device 34 may be adapted to be advanced over the guidewire 26 in combination with the first elongate medical device 32 in order to create or enlarge an aperture formed in the atrial septum 18.

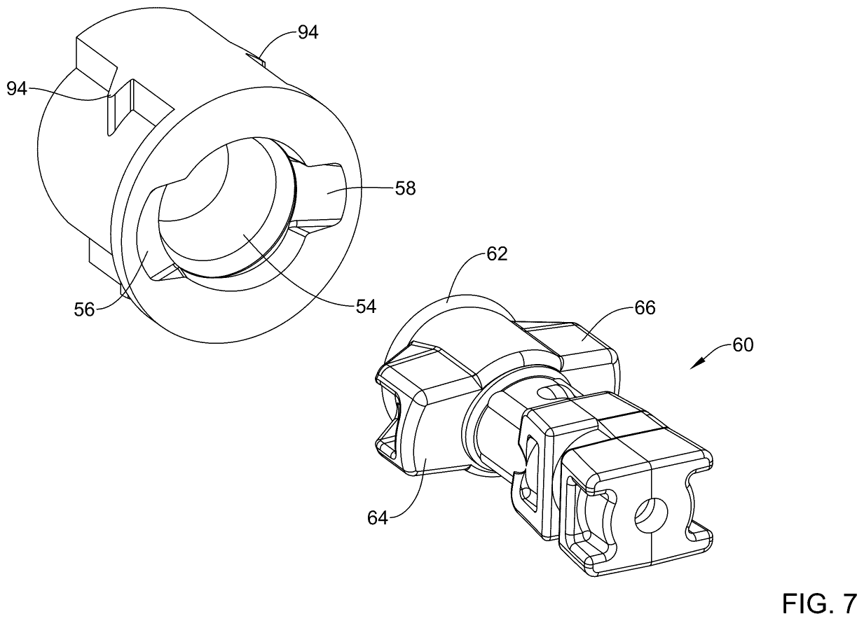

In some cases, the first proximal hub 42 includes an aperture 54 that is adapted to accommodate portions of the second proximal hub 50. In some cases, the aperture 54 forms a first pattern. As an example, the aperture 54 may include one or more wings that extend radially outwardly from the aperture 54. As shown, there is a first wing 56 that extends in a first radial direction from the aperture 54 and a second wing 58 that extends in a second radial direction from the aperture 54. In some cases, the first wing 56 may extend in a radial direction that is about 180 degrees circumferentially separated from the radial direction in which the second wing 58 extends. In some cases, the first wing 56 and the second wing 58 may extend in first and second directions, respectively, that are not about 180 degrees apart. In some cases, the first wing 56 may have a first width and the second wind 58 may have a second width that is different from the first width. As shown, more pronouncedly in FIG. 7, the first width is greater than the second width. As an alternative, the first width may be less than the second width. Having different widths for the first wing 56 and the second wing 58 contributes to providing a unidirectional rotational lock. In some cases, having the first wing 56 and the second wing 58 circumferentially spaced apart less than 180 degrees can contribute to providing a unidirectional rotational lock without requiring that the first width and the second width are different.

In some cases, the second proximal hub 50 includes an extension member 60 that is adapted to engage the aperture 54 that is formed within the first proximal hub 42. The extension member 60 may include a second pattern that is complementary to the first pattern. The first pattern and the second pattern may be adapted to only allow the extension member 60 to fit into the aperture 54 in a single orientation relative to the first proximal hub 42. As an example, the extension member 60 may include a round portion 62 that is adapted to be inserted into the aperture 54. The extension member 60 includes a first tab 64 that extends in a first radial direction from the round portion 62 and a second tab 66 that extends in a second radial direction from the round portion 62. As shown, the first tab 64 may extend in a radial direction that is about 180 degrees circumferentially separated from the radial direction in which the second tab 66 extends. In some cases, the first tab 64 and the second tab 66 may extend in first and second directions, respectively, that are not 180 degrees apart. In some cases, the first tab 64 and the second tab 66 may extend in first and second directions, respectively, that match the respective directions in which the first wing 56 and the second wing 58 extend from the aperture 54. In some cases, the first tab 64 may have a first width and the second tab 66 may have a second width that is different from the first width. As shown, more pronouncedly in FIG. 7, the first width is greater than the second width. As an alternative, the first width may be less than the second width.

In order to facilitate a unidirectional rotational lock between the first proximal hub 42 and the second proximal hub 50, the relative orientation and dimensions of the first tab 64 matches the relative orientation and dimensions of the first wing 56 and the relative orientation and dimensions of the second tab 66 matches the relative orientation and dimensions of the second wing 58. It will be appreciated that the dimensions may include not only width, but length and height as well. The structure of the aperture 54 and the structure of the extension member 60 cooperate to enable the second elongate medical device 34 to be inserted into and extend through the first elongate medical device 32 with the second elongate medical device 34 having a single orientation relative to the first elongate medical device 32. That way, any curvature within the distal region 48 of the second elongate shaft 44 will be appropriately orientated with respect to any curvature within the distal region 40 of the first elongate shaft 36.

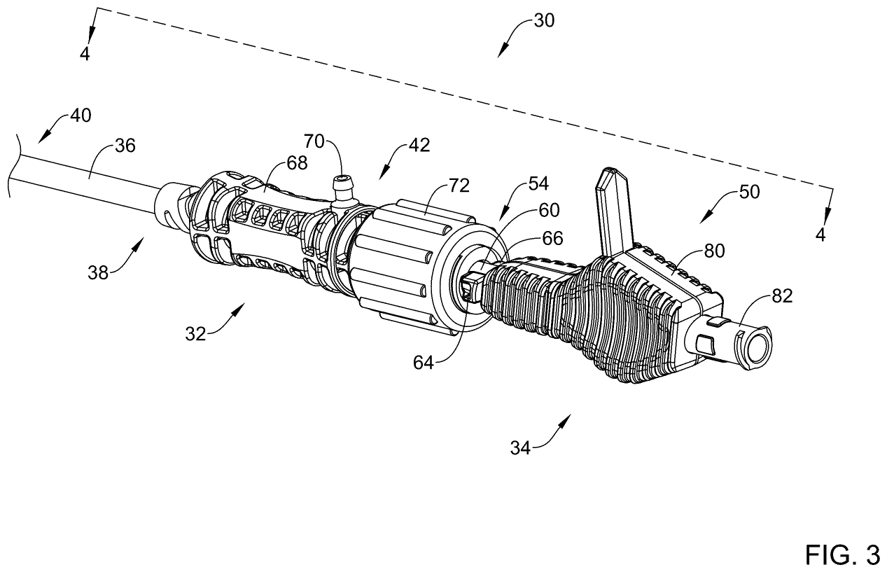

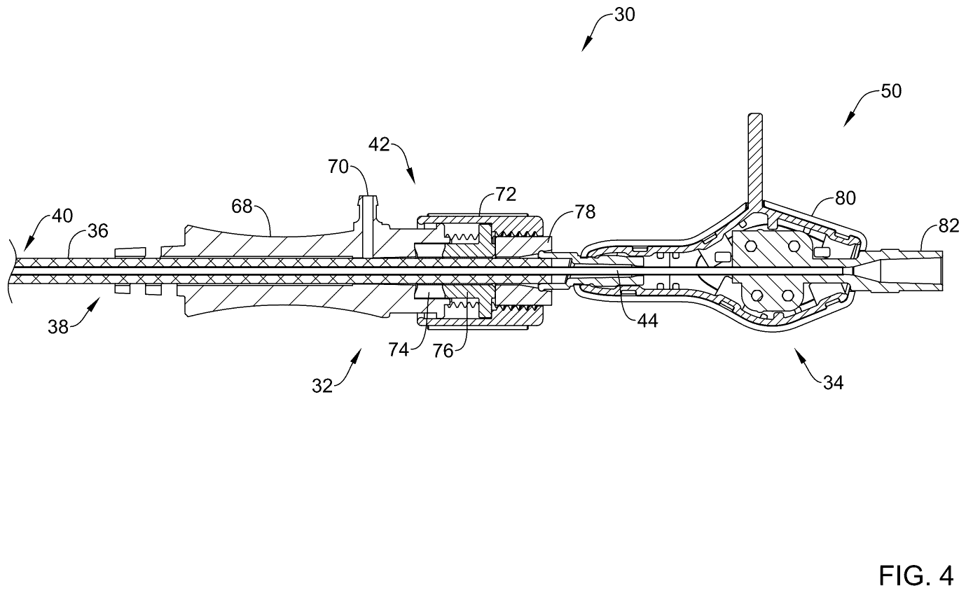

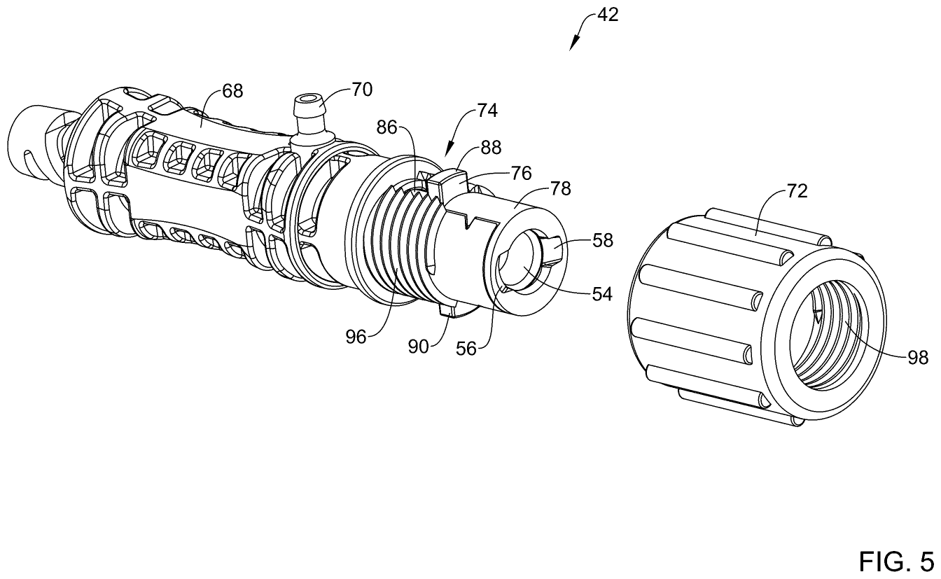

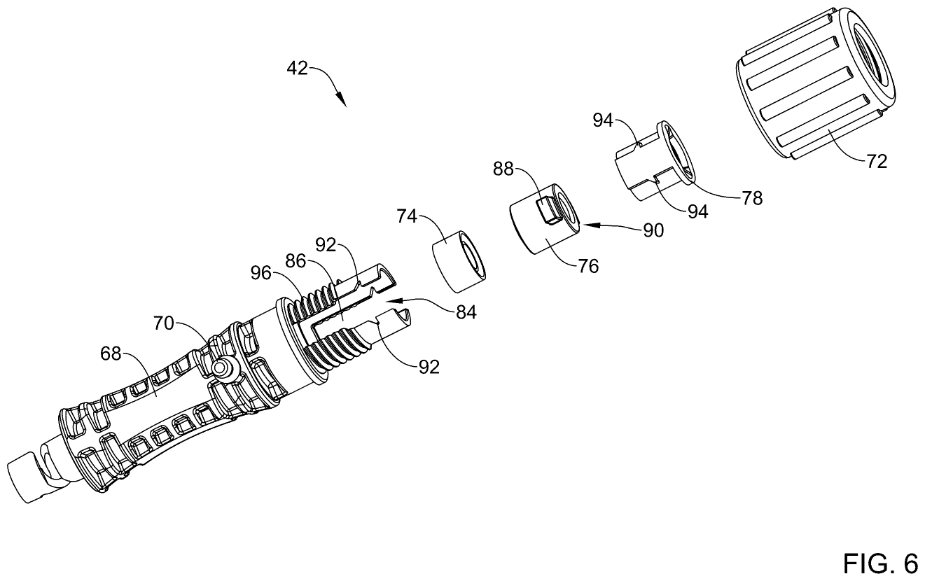

FIG. 3 is a perspective view of the medical device assembly 30, showing the second elongate medical device 34 disposed within and extending through the first elongate medical device 32. FIG. 4 is a cross-sectional view taken along the line 4-4 of FIG. 3. The first proximal hub 42 includes a hub body 68. In some cases, the first proximal hub 42 may include a flushing port 70. A knurled knob 72 may include an internal threaded surface that is adapted to engage a corresponding external threaded surface on the hub body 68. A seal 74 is disposed within the hub body 68. The seal 74 may be an elastomeric seal, for example. A pusher 76 is also disposed within the hub body 68 and is positioned to engage and compress the seal 74 as the knurled knob 72 is rotated in a direction that causes the knurled knob 72 to translate in a distal direction relative to the hub body 68. In some cases, the pusher 76 is held in a specific rotational orientation relative to the hub body 68, as will be shown in FIGS. 5 and 6. The first proximal hub 42 includes an insert 78 that includes the aperture 54 and the first and second wings 56 and 58, respectively. The insert 78 may be held within the hub body 68 in a specific rotational orientation relative to the hub body 68, thereby fixing the relative rotational orientation of the first wing 56 and the second wing 58 relative to the hub body 68. Since the hub body 68 is fixedly secured to the first elongate shaft 36, this maintains a specific rotational orientation of the first proximal hub 42 (and hence the second proximal hub 50) relative to the distal region 40 of the first elongate shaft 36.

The second proximal hub 50 includes a hub body 80. In some cases, the hub body 80 includes an outer surface that provides a graspable profile that makes it easy for a physician or other professional to grasp the second proximal hub 50. The hub body 80 may be a clamshell having a first half and a second half that are snap fit together, for example. The second elongate shaft 44 extends distally through the hub body 80 and terminates in a fitting 82 that may be used for attaching other devices.

FIGS. 5 and 6 are exploded views of the first proximal hub 42. As can be seen, the hub body 68 includes a hollow 84 that houses the seal 74, the pusher 76 and the insert 78. The hub body 68 includes a pair of elongate slots 86 that accommodates a first tab 88 and a second tab 90 that extend outwardly from the pusher 76. The first tab 88 and the second tab 90 are circumferentially separated by about 180 degrees, and fit into each of the pair of elongate slots 86 such that a relative rotational orientation of the pusher 76 relative to the hub body 68 is fixed. The hub body 68 also includes several indentations 92 that accommodate corresponding tabs 94 that are formed on the insert 78 such that the relative rotational orientation of the insert 78 relative to the hub body 68 is fixed.

An outer surface of the hub body 68 includes an external threaded surface 96 that is adapted to engage a corresponding internal threaded surface 98 disposed within the knurled knob 72. In some cases, the internal threaded surface 98 of the knurled knob 72 may be axially offset from the external threaded surface 96 of the hub body 68 such that the knurled knob may be rotated without the internal threaded surface 98 engaging the external threaded surface 96 unless the knurled knob 72 is translated in a distal direction. Once the internal threaded surface 98 has engaged the external threaded surface 96, rotation of the knurled knob 72 in an appropriate rotational direction will cause the knurled knob 72 to urge the pusher 76 in a distal direction, thereby compressing the seal 74.

FIG. 7 is an exploded view of the insert 78 and the extension member 60, illustrating how the first tab 64 is aligned with the first wing 56 and the second tab 66 is aligned with the second wing 58. This allows the second elongate medical device 34 to be inserted into the first elongate medical device 32 in a single rotational orientation. The second elongate medical device 34 can only be inserted into the first elongate medical direction 32 in a single rotational orientation. As a result, any curvature within the distal region 40 of the first elongate shaft 36 is appropriately orientated with respect to any curvature within the distal region 48 of the second elongate shaft 44. In some cases, any curvature within the distal region 48 of the second elongate shaft 44 may be used to temporarily straighten any curvature within the distal region 40 of the first elongate shaft 36. In some cases, any curvature within the distal region 48 of the second elongate shaft 44 may be used to temporarily enhance or strengthen any curvature within the distal region 40 of the first elongate shaft 36.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, The devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device assembly for accessing a left atrial appendage, the medical device assembly comprising:
a first elongate medical device comprising:
a first elongate shaft having a first proximal region and a first distal region; and
a first proximal hub secured to the first proximal region; and
a second elongate medical device comprising:
a second elongate shaft having a second proximal region and a second distal region; and
a second proximal hub secured to the second proximal region;
wherein the first elongate medical device is adapted to form a releasable unidirectional rotational lock with the second elongate medical device.

2. The medical device assembly of claim 1, wherein the second elongate shaft is adapted to fit within a lumen extending through the first elongate shaft.

3. The medical device assembly of claim 2, wherein the second proximal hub is adapted to releasably couple to the first proximal hub when the second elongate shaft is disposed within the lumen extending through the first elongate shaft.

4. The medical device assembly of claim 3, wherein the second proximal hub is adapted to form a releasable unidirectional rotational lock with the first proximal hub.

5. The medical device assembly of claim 1, wherein the first elongate medical device is adapted to be advanced over a guidewire in order to reach an atrial septum.

6. The medical device assembly of claim 5, wherein the second elongate medical device is adapted to be advanced over the guidewire in combination with the first elongate medical device in order to create or enlarge an aperture formed in the atrial septum.

7. The medical device assembly of claim 1, wherein:
the first proximal hub includes an aperture forming a first pattern; and
the second proximal hub includes an extension member adapted to extend into the aperture, the extension member including a second pattern complementary to the first pattern.

8. The medical device assembly of claim 7, wherein the first pattern and the second pattern are adapted to only allow the extension member to fit into the aperture in a single orientation relative to the first proximal hub.

9. The medical device assembly of claim 1, wherein the first elongate medical device comprises a guide catheter.

10. The medical device assembly of claim 1, wherein the second elongate medical device comprises an elongate dilator.

11. The medical device assembly of claim 10, wherein the second proximal hub comprises a graspable profile to facilitate advancement of the second elongate medical device into the first elongate medical device.

12. A medical device assembly for accessing a left atrial appendage, the medical device assembly comprising:
a guide catheter comprising:
a guide catheter shaft; and
a guide catheter hub secured to a proximal region of the guide catheter shaft; and
a dilator comprising:
a dilator shaft; and
a dilator hub secured to a proximal region of the dilator shaft;
wherein the dilator can only be inserted into the guide catheter with the dilator hub in a specific rotational orientation relative to the guide catheter hub.

13. The medical device assembly of claim 12, wherein the guide catheter hub further comprises a threaded knob that when rotated is adapted to urge the dilator further distally after the dilator has been inserted into the guide catheter with the dilator hub in the specific rotational orientation relative to the guide catheter hub.

14. The medical device assembly of claim 12, wherein the guide catheter hub further comprises an aperture adapted to accommodate the dilator hub, the aperture including one or more wings extending radially outwardly from the aperture.

15. The medical device assembly of claim 14, wherein the dilator hub further comprises an extension member having one or more tabs that extend radially outwardly from the extension member, the one or more tabs complementary to the one or more wings.

16. The medical device assembly of claim 12, wherein the guide catheter is adapted to be advanced over a guidewire in order to reach an atrial septum.

17. The medical device assembly of claim 16, wherein the elongate dilator is adapted to be advanced over the guidewire in combination with the guide catheter in order to create or enlarge an aperture formed in the atrial septum.

18. The medical device assembly of claim 16, wherein the dilator hub comprises a graspable profile to facilitate advancement of the dilator into the guide catheter.

19. A medical device assembly for accessing a left atrial appendage, the medical device assembly comprising:
a guide catheter comprising:
a guide catheter shaft; and
a guide catheter hub secured to a proximal region of the guide catheter shaft, the guide catheter hub including an aperture having one or more wings extending radially outwardly from the aperture; and
a dilator comprising:
a dilator shaft;
a dilator hub secured to a proximal region of the dilator shaft; and
an extension member extending distally from the dilator hub, the extension member including one or more tabs extending radially outwardly from the extension member, the one or more tabs adapted to fit into the one or more wings.

20. The medical device assembly of claim 19, wherein the one or more wings and the one or more tabs are adapted to permit the extension member to fit into the aperture in only one relative rotational orientation between the guide catheter hub and the dilator hub.
